(19) United States
(12) Patent Application Publication
(10) Pub. No.: US 2026/0114918 A1
(43) Pub. Date: Apr. 30, 2026

(54) SHEATH WITH INTEGRATED CONDUCTOR WIRE

(71) Applicant: Boston Scientific Scimed, Inc., Maple Grove, MN (US)

(72) Inventors: Miles Montgomery, Holland Landing (CA); Palak Patel, Toronto (CA); Natalie Cichon, Toronto (CA); Yara Hosein, Mississauga (CA); Ros Thomas Brannick, Picton (CA); Matthew Nelson Frost, New Brighton, MN (US)

(21) Appl. No.: 19/003,679

(22) Filed: Dec. 27, 2024

Related U.S. Application Data
(60) Provisional application No. 63/616,402, filed on Dec. 29, 2023.

(51) Int. Cl. A61B 18/14; A61B 17/00; A61B 18/00; A61L 29/08; A61M 25/00

(52) U.S. Cl. CPC A61B 18/1492; A61L 29/085; A61B 2017/00526; A61B 2018/00077; A61B 2018/00136; A61B 2018/00577; A61M 25/0012; A61M 25/0045

(57) ABSTRACT

Sheaths and methods of manufacturing the sheaths are disclosed. An example sheath includes an elongated shaft defining a lumen along a longitudinal axis and having a distal section opposite a proximal section. The elongated shaft includes an outermost elongated support member coaxial with the lumen. The support member includes an outer side. A lead conductor is wound around the outer side of the support member at the distal section. A cover member is disposed on the lead conductor and the support member, the cover member and lead conductor. A conductive portion of the lead conductor is exposed at a selected axial location in the distal section. An electrode is swaged against the cover member at the selected axial location and in electrical connection with the exposed conductive portion via mechanical compression.

### CROSS REFERENCE TO RELATED APPLICATIONS

This application claims priority to U.S. Provisional Ser. No. 63/616,402 entitled “SHEATH WITH INTEGRATED CONDUCTOR WIRE,” filed Dec. 29, 2023, which is hereby incorporated by reference in its entirety.

### TECHNICAL FIELD

The present disclosure relates to medical devices for catheterization procedures, such as medical devices for electrophysiological procedures. More specifically, the present disclosure relates to catheter elements, such as sheaths, and methods for manufacturing sheaths.

### BACKGROUND

Various medical fields use different types of catheters to achieve access to a physiological site in medical procedures. For instance, electrophysiological procedures involve guiding catheters elements into the heart and tracking the location of the catheter elements with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths, dilators, and other medical tools, which can be referred to as catheter elements in this disclosure. Electrophysiological procedures can involve the visualization of the heart, heart activity, and the position of the catheter elements within the heart. A common visualization system involves the use of fluoroscopy, which can expose the patient and clinician to ionizing radiation. Electroanatomical mapping is an alternative visualization technique that does not involve the use of radiation.

Electroanatomical mapping allows a clinician to accurately determine the location of an arrhythmia, define cardiac geometry in three dimensions, delineate areas of anatomic interest, and permits imaging of the catheter elements for positioning and manipulation. Catheter elements used with electroanatomical mapping systems can include tracking capabilities, such as navigation enabled or impedance-based tracking methodologies. Navigation-enabled catheters use magnetic sensors in the presence of magnetic fields to track the location and orientation of the catheters. But not all catheter elements include magnetic sensors. Impedance-based catheter elements, such as sheaths, use electrodes in the presence of electric fields to track the sheaths.

### SUMMARY

In Example 1, a sheath, comprising an elongated shaft defining a lumen along a longitudinal axis and having a distal section opposite a proximal section, the elongated shaft having an outermost elongated support member coaxial with the lumen, the support member having an outer side; a lead conductor wound around the outer side of the support member at the distal section; a cover member disposed on the lead conductor and the support member, the cover member and lead conductor, a conductive portion of the lead conductor exposed at a selected axial location in the distal section; and an electrode swaged against the cover member at the selected axial location and in electrical connection with the exposed conductive portion via mechanical compression.

In Example 2, the sheath of Example 1, wherein the support member includes a braided fabric comprising a plurality of fibers braided together.

In Example 3, the sheath of any of Examples 1 and 2, wherein the lead conductor is wound on the outer side of the support member.

In Example 4, the sheath of Example 3, wherein the lead conductor is coiled on the outer side of the support member.

In Example 5, the sheath of Example 3, wherein the lead conductor includes a plurality of lead conductors, and the plurality of lead conductors are braided on the outer side of the support member.

In Example 6, the sheath of Example 2, wherein the lead conductor is a fiber of the plurality of fibers braided together to form the braided fabric.

In Example 7, the sheath of any of Examples 1-6, wherein the support member is conductive and further comprising an electrically insulative sleeve disposed on the support member and radially underneath the exposed conductive portion.

In Example 8, the sheath of Example 7, wherein the electrically insulative sleeve is a polyethylene terephthalate heat shrink.

In Example 9, the sheath of any of Example 1-8, wherein the lead conductor includes a conductive wire covered in an electrically insulative sheath member, and the exposed conductive portion includes a bare wire portion.

In Example 10, the sheath of any of Examples 1-9, wherein the lead conductor includes a conductive element having a triangular cross section with a vertex facing radially outwardly and a side facing radially inwardly.

In Example 11, the sheath of any of Examples 1-10, wherein the electrode is ring electrode.

In Example 12, the sheath of Example 11, wherein the ring electrode includes an inner diameter, and the cover member includes an outer diameter, wherein the inner diameter is greater than the outer diameter prior to the electrode being swaged against the cover member.

In Example 13, the sheath of any of Examples 1-12, wherein the shaft includes one support member.

In Example 14, the sheath of any of Examples 1-13, wherein the electrode is not welded to the lead conductor.

In Example 15, the sheath of any of Examples 1-14, wherein the sheath is a guide sheath having a proximal end coupled to a handle.

In Example 16, a sheath, comprising an elongated shaft defining a lumen along a longitudinal axis and having a distal section opposite a proximal section, the elongated shaft having an outermost elongated support member coaxial with the lumen, the support member having an outer side; a lead conductor wound around the outer side of the support member at the distal section; a cover member disposed on the lead conductor and the support member, the cover member and lead conductor, a conductive portion of the lead conductor exposed at a selected axial location in the distal section; and an electrode swaged against the cover member at the selected axial location and in electrical connection with the exposed conductive portion via mechanical compression.

In Example 17, the sheath of Example 16, wherein the support member includes a braided fabric comprising a plurality of fibers braided together.

In Example 18, the sheath of Example 16, wherein the lead conductor is wound on the outer side of the support member.

In Example 19, the sheath of Example 18, wherein the lead conductor is coiled on the outer side of the support member.

In Example 20, the sheath of Example 18, wherein the lead conductor includes a plurality of lead conductors, and the plurality of lead conductors are braided on the outer side of the support member.

In Example 21, the sheath of Example 17, wherein the lead conductor is a fiber of the plurality of fibers braided together to form the braided fabric.

In Example 22, the sheath of Example 16, wherein the support member is conductive and further comprising an electrically insulative sleeve disposed on the support member and radially underneath the exposed conductive portion.

In Example 23, the sheath of Example 22, wherein the electrically insulative sleeve is a polyethylene terephthalate heat shrink.

In Example 24, the sheath of Example 16, wherein the lead conductor includes a conductive wire covered in an electrically insulative sheath member, and the exposed conductive portion includes a bare wire portion.

In Example 25, the sheath of Example 16, wherein the lead conductor includes a conductive element having a triangular cross section with a vertex facing radially outwardly and a side facing radially inwardly.

In Example 26, the sheath of Example 16, wherein the electrode is ring electrode.

In Example 27, the sheath of Example 26, wherein the ring electrode includes an inner diameter, and the cover member includes an outer diameter, wherein the inner diameter is greater than the outer diameter prior to the electrode being swaged against the cover member.

In Example 28, the sheath of Example 26, wherein the ring electrode is a contiguous ring electrode.

In Example 29, the sheath of Example 16, wherein the shaft includes one support member.

In Example 30, the sheath of Example 16, wherein the electrode is not welded to the lead conductor.

In Example 31, the sheath of Example 16, wherein the sheath is a guide sheath having a proximal end coupled to a handle.

In Example 32, a sheath, comprising an elongated shaft defining a lumen along a longitudinal axis and having a distal section opposite a proximal section, the elongated shaft having an outermost elongated support member coaxial with the lumen, the support member having an outer side; a lead conductor wound around the outer side of the support member at the distal section; a cover member disposed on the lead conductor and the support member, the cover member and lead conductor skived at a selected axial location in the distal section to expose a conducive portion of the lead conductor; and an electrode swaged against the cover member at the selected axial location and in electrical connection with the exposed conductive portion via mechanical compression.

In Example 33, the sheath of Example 32, wherein the support member includes a braided fabric comprising a plurality of fibers braided together, and wherein the lead conductor is one of wound on the outer side of the support member and the lead conductor is a fiber of the plurality of fibers braided together to form the braided fabric.

In Example 34, a method for forming a sheath, the method comprising: providing an elongated shaft defining a lumen along a longitudinal axis and having a distal section opposite a proximal section, the elongated shaft having an outermost elongated support member coaxial with the lumen, the support member having an outer side; winding a lead conductor around the outer side of the support member at the distal section; disposing a cover member disposed on the lead conductor and the support member; exposing a conductive portion of the lead conductor at a selected axial location in the distal section; and swaging an electrode against the cover member at the selected axial location and in electrical connection with the exposed conductive portion via mechanical compression.

In Example 35, the method of Example 34, wherein the cover member and lead conductor are skived to expose the lead conductor at the selected axial location in the distal section.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, the example clinical setting having an example electrophysiology system.

FIG. 2 is a schematic diagram illustrating an example sheath that can be used with the example electrophysiology system. of FIG. 1.

FIG. 3 is a schematic diagram illustrating from a side view a feature of an embodiment of the sheath of FIG. 2.

FIG. 4 is a block diagram illustrating an example method of manufacturing the sheath of FIG. 2.

FIGS. 5A-5D are schematic diagrams illustrating from a side view multiple stages of manufacturing the sheath of FIG. 2.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

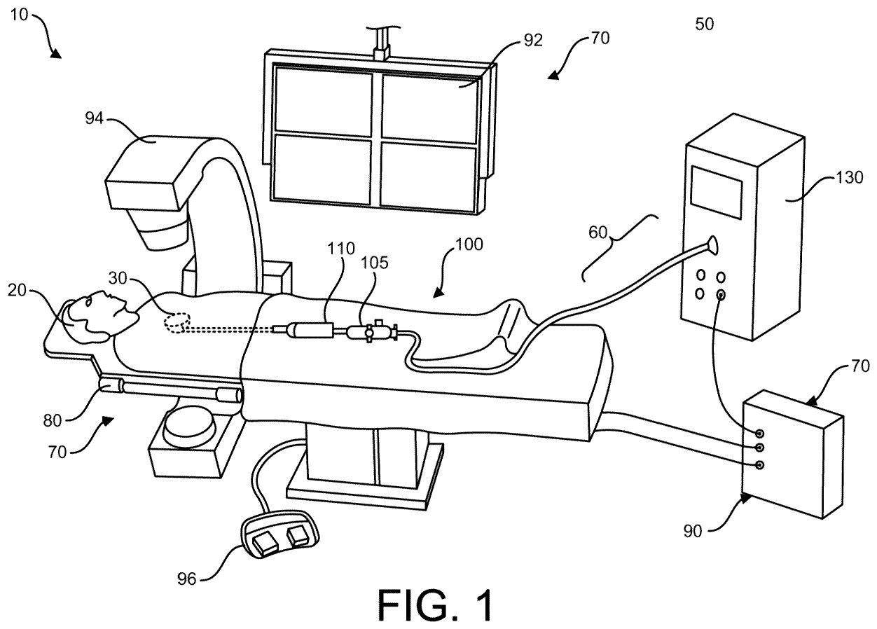

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which in the example includes catheter elements such as an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 and sheath 110 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheter elements. For instance, a catheter element defines a longitudinal axis that passes through a centroid of a cross section of the catheter element, such as the centroid of a cross section of a shaft of catheter 105 or a centroid of a cross section of a main lumen of a sheath 110. In the example, the catheter 105 is disposed within the sheath 110. The catheter 105 and sheath 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative embodiment, the EAM system 70 can include the OPAL™ HDx mapping system marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the catheter 105 and the sheath 110, generate an output that can be processed by the mapping and navigation controller 90 to track the location and orientation of the sensor or sensors, and consequently, the corresponding device, within the localization volume. In the illustrated embodiment, the device tracking of the catheter 105 is accomplished using magnetic tracking techniques, in which the field generator 80 is a magnetic field generator that generates a magnetic field defining the localization volume, and location sensors on the tracked devices are magnetic field sensors.

In other embodiments, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter and associated sheath 110, or both. In these examples, the location sensing elements can constitute tracking electrodes on the tracked sheaths that generate outputs received and processed by the mapping and navigation controller 90 to track the location of the various location sensing electrodes within the localization volume. For instance, an impedance tracking methodology can employ the use of a patch electrode (not show) attached to the patient's body, a current or impedance based can be determined between the tracking electrode on the catheter and the patch electrode and the sheath and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the catheter 105 or another catheter, sheath, or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters and sheaths include tracking electrodes disposed on the deflectable portions of the respective shafts. Multiple tracking electrodes can be employed on the deflectable portions of the catheters and sheaths for the EAM system 70 to detect and recreate the curvature of the catheter elements s in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Several constraints are employed in the design and implementation of tracking electrodes and associated lead conductors. Among these constraints include that each tracking electrode and associated lead conductor are to be electrically isolated from one another as well as from other conductive material in the sheath such as a conductive braided member along the length of the shaft. Additionally, precise placement of the tracking electrode on the shaft is desired. For instance, the tracking electrode can be radiopaque, and clinicians can use the tracking electrode to visualize placement of the sheath while using fluoroscopy. Also, electrode location with respect to the catheter elements and diameter and interelectrode spacing are programmed parameters in several tracking and mapping software programs, and three-dimensional reconstruction and modeling is performed with electrode spacing as a constraint in the modeling curve. Thus, the design and implementation of sheaths using tracking electrodes include lumens with ready access to carry conductor leads.

The manufacture of such sheaths, however, can be cumbersome and expensive. For example, the manual process to currently incorporate a lead wire along the shaft to the tracking electrode presents a bottleneck in manufacturing. Wire stringing is subject to poor ergonomics. Sheaths can include four electrodes (or more), and each electrode is associated with a separate lead conductor. Lead wires can be included in elongate lumens that transition from under to over a braided support member, which creates complex downstream processing. Braiding, braid terminations, distal coiling, hole drilling, and sealing holes to cover potential leak paths are often present in manufacturing. Further, the parts and manufacturing processes are expensive. Additional joining of the electrode and the lead wire via laser or resistance weld adds complexity and if performed by an external vendor can account for substantial charges. Further, the weld is subject to potential stresses during manufacturing assembly potentially leading to a fragile or weakened joint, and precise controls are used to ensure welding of the wire-electrode outputs are capable of meeting outputs.

This disclosure is directed to catheter elements, such as sheaths and methods for assembling catheter elements that include a conductive lead for a tracking electrode wound around a support member and under a cover member. For instance, the lead conductors are coiled or braided over the support member or included with the braided fibers of a braided support member and coated with an electrically insulate material to form the cover member. The cover member and lead conductor expose a portion of bare wire, such as the bare wire is ablated or skived, at an axial location along the side of a support member on a shaft. The electrode is swaged against the shaft to make a robust electrical connection with the lead conductor. The sheaths are manufactured without drilling holes in the support member to extend the lead conductors, or adding expensive tubes to carry the lead conductors, or using expensive laser welds to make electrical connections.

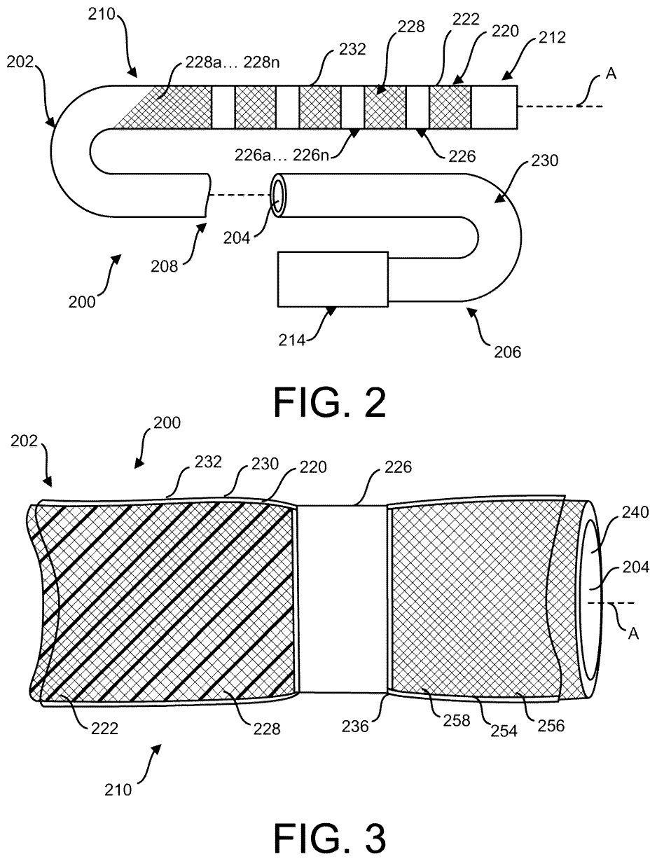

FIG. 2 illustrates an embodiment of a catheter element 200, such as sheath 200, that can be used in the example clinical setting 10 in catheter assembly 100. For instance, sheath 200 can be further configured as a guide catheter, dilator, or other flexible, deflectable medical tool that can be tracked via an impedance tracking system such as EAM system 70. The illustrated sheath 200 includes an elongated shaft 202, such as an elongated and flexible shaft 202 defining a longitudinal axis A. The shaft 202 also defines a main lumen 204 along the longitudinal axis A and has a proximal section 206, a longitudinal section 208, and a distal section 210 opposite the shaft 202 from the proximal section 206. The distal section 210 includes a distal tip section 212. The proximal section 206 can be coupled to a handle 214 proximal to the shaft 202. An elongated and flexible support member 220 is disposed over some or all of the shaft 202 or included in the shaft 202 along the longitudinal axis A and coaxial with the lumen 204. In various embodiments, the support member 220 is a flexible braid (e.g., stainless steel braid or a high-strength polymer braid, or braid formed from a combination of materials), a coiled shaft, a hypotube (e.g., a laser cut hypotube), or a high-density polyethylene (HDPE) tube. The support member 220 includes an outer surface 222.

The sheath 200 includes a plurality of other components disposed along the shaft 202, such as on the distal section 210. In the illustrated embodiment, the sheath 200 includes a tracking electrode 226, such as a plurality of tracking electrodes 226a . . . 226n, on the distal section 210 of the shaft 202. The tracking electrodes 226 are illustrated as ring electrodes mechanically coupled to the shaft 202. Each tracking electrode 226a . . . 226n is electrically coupled to a respective lead conductor 228a . . . 228n wound around the outer surface 222 along the support member 220 and terminated at the proximal section 206. In the example, the tracking electrodes 226a . . . 226n are configured for use with an impedance-based tracking system to detect the position of the shaft 202, such as the distal tip section 212. In some embodiments, the tracking electrodes 226a . . . 226n are also radiopaque. In embodiments in which the support member 220 includes conductive materials, the lead conductor 228 is covered in an electrically insulative sheath or an electrically insulative layer is disposed between the lead conductor 228 and the support member 220 to reduce the likelihood of an electrical short between the lead conductor 228 and the support member 220.

The proximal section 206 or handle 214 can include an electrical connection that can be coupled to an impedance-based tracking system, such as EAM system 70. In one example, the electrical connection is available under the trade designation LEMO. The sheath 200 further includes a cover member 230 disposed over the support member 220 and lead conductor 228 to form an outer surface 232 of the sheath 200.

The sheath 200 can include additional components for a selected implementation. In some embodiments, such as in the case of the sheath 200 configured as a guide sheath or dilator, the proximal section 206 or handle 214 can include a hemostatic valve that can be coupled to a source of irrigation fluid and a port to receive catheter elements, such ablation catheters within the main lumen 204. In some embodiments, the distal tip section 212 can be configured as a dilator tip. In some embodiments, the shaft 202 can include a liner layer (not shown) underneath the support member 220 and coaxial with the main lumen 204. In other embodiments, the liner layer is the support member 220, and the shaft 202 does not include a braided member or other member between the liner layer and the elongated tube 222. The liner layer can define an inner wall of the main lumen 204. In examples such as in which the sheath 200 is a guide sheath or dilator, the liner layer can be a thin wall constructed of polytetrafluoroethylene (PTFE). In some examples, such as in the case of the catheter element configured as an ablation catheter, the proximal portion 206 or handle can be coupled to a source of ablation energy such as an electrical signal from console 130 or a cryogenic fluid. For instance, the main lumen 204 can be configured to carry other electrical leads, such as leads to other sensors, steering wires, or a conduit of irrigation fluid along the shaft 202 to the distal portion 210. In some examples, the distal tip section 212 can be configured to include other sensors.

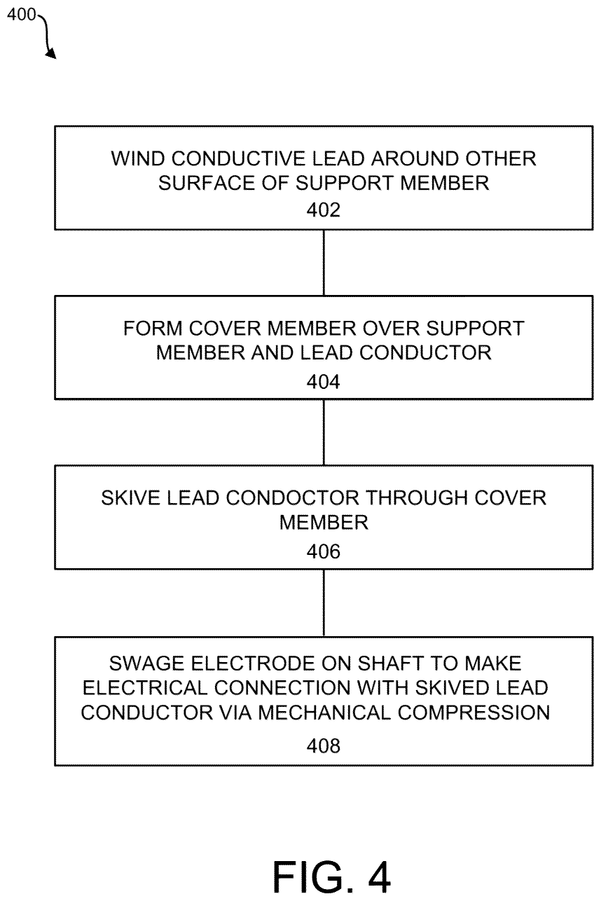

FIG. 3 illustrates features of an embodiment of the sheath 200, such as the distal section 210 proximal to the distal tip 212. In particular, the sheath 200 includes the elongated and flexible shaft 202. The shaft 202 includes the elongated and flexible support member 220 and cover member 230, which are coaxial with the main lumen 204. In some embodiments, the shaft 202 includes liner layer 240 also coaxial with the main lumen 204 and defining an inner wall of the main lumen 204. The support member 220 can be disposed on the liner layer 240, for example, such as disposed directly on the liner layer 240. The support member 220 includes an inner side or surface (not shown) disposed toward the main lumen 204, such as on liner layer 240, and an outer side or outer surface 222 disposed opposite the inner side and the main lumen 204. In some embodiments, the shaft 202 can include a plurality of concentric or coaxially support members, such an innermost support member and an outermost support member 220. In the example, a single support member 220 is also an outermost support member 220.

The support member 220 is illustrated in the embodiment as a braided member 220, which can provide characteristics to the sheath 200 such as reduced kinking, wrinkling, or buckling of the shaft 202 and can provide enhanced balance for pushability, deflectability, and torque transmission such as during rotation about the longitudinal axis A. In another embodiment, the support member 220 can include a coiled shaft member, a laser cut hypotube, just the liner layer 240, or other elongated shaft material such as HDPE. In the illustration, the braided member as the support member 220 is constructed from a woven fabric 254 or layer of braided strands or fibers 256 that form interstitial spaces 258 between the fibers 256. The braided member as the support member 220 can further be characterized by the warp and weft and bias of the fibers 256 as well as picks per inch. For instance, in some embodiments, the picks per inch can remain generally uniform along the entire longitudinal length of the braided member as the support member 220. In some embodiments, the picks per inch can vary along portions of the longitudinal length of the braided member 220. The braided member as the support member 220 can be constructed from fibers 256 that include stainless steel fibers, such as conductive fibers, or high strength polymer fibers, or as layers of different materials. Additionally, the number of strands, braid ends carriers per strand, and braid pattern can vary as well as the use of a single filament or multifilament strands. In some examples, the support member 220 is configured as a coiled member.

The sheath 200 includes a lead conductor 228 wound around the outer surface 222 of the support member 220. For instance, the lead conductor 228 is wound on a fully formed support member 220. For example, the lead conductor 228 is wound on top of the braided member of the outermost support member 220. In another example, the lead conductor is wound on top of an outer surface of a hypotube. In these examples, the lead conductor 228 is disposed between the support member 220 and cover 230. In the illustrated example, the lead conductor 228 is wound as a coil, i.e., the lead conductor 228 is coiled on the support member 220. In other examples, a plurality of lead conductors, such as lead conductors 228a . . . 228n, are wound as a braid, i.e., the lead conductors 228a . . . 228n are braided together, on top of the support member 220. In still another embodiment, the lead conductors are wound on the support member 220 as braided into the braided support member 220. For instance, the lead conductor 228 is wound into the support member 220 as part of the fibers 256 forming the support member 220. The lead conductor 228 comprises the woven fabric 254 and is included as the warp and weft and picks per inch of the fibers 256 of support member 220.

The coiled lead conductors and braided lead conductors can enhance mechanical properties of the shaft 202, such as torque or kink resistance and ovalization resistance during deflection. The wound lead conductor 228, however, is subject to strain and separation from the support member 220 in steerable or bendable sheath 200. For example, wide pitched lead conductor coils can separate from the support member 220 in various locations along the shaft 202 after the shaft 202 has been bent. Narrow pitched lead conductor coils have been demonstrated to resists separation and provide sufficient strain relief. Accordingly, in steerable or bendable sheaths, the pitch of wound lead conductor, whether coiled or braided, is approximately the pitch of the braided support member 220.

In the illustrated embodiments, the lead conductor 228 includes a conductive wire covered in an electrically insulative sheath member to electrically isolate the wire from other conductive elements in the shaft 202 rather than a bare wire. For example, the sheath member electrically isolates the lead conductor 228 from a conductive braided member, other lead conductors, and electrodes 226 not associated with the lead conductor 228 as well as connection elements on the proximal section 206 of the sheath 200. The lead conductor 228 includes an exposed conductive portion (not shown), such as an exposed bare wire having the sheath removed, in a location proximate the associated electrode 226 to make an electrical connection with the associated electrode. The lead conductor 228 also include an exposed conductive portion at the proximal section 206 to make electrical contact with connective elements. In some embodiments, the other conductive elements include electrically isolating features or, in the example of the support member, are made from non-conductive materials and the lead conductor 228 can be a bare wire. In one example, the cross-sectional shape of a conductive element (wire) of the lead conductor 228 is round or curvilinear, such as circular or oval. In another example, the cross-section shape of the conductive element (wire) of the lead conductor 228 is triangular having a vertex facing radially outwardly and an opposite side facing radially inwardly against the support member 220. The vertex is configured to cut through the cover member 230 to contact the electrode 226.

The cover member 230, or outer layer, is disposed on the support member 220 and the conductive leads 228a . . . 228n. In some embodiments, the cover member 230 can be formed as a coating of an electrically insulative reflowable plastic or thermoplastic material that extends over the support member 220 and conductive leads 228a . . . 228n and seals underlying components of the shaft 202. For instance, the coating can seep by reflowing over braided material of the support member 220 such as over the fibers 256 and conductive leads 228a . . . 228n and into the interstitial spaces 258 of the braided material of the support member 220. In one example, the cover member 230 is a polyether block amide and, in some examples, is available under the trade designations PEBAX from Arkema, S.A., and VESTAMID E from Evonik Industries, AG. The cover member 230 and lead conductor 228 are skived or ablated at a selected axial location of the shaft 202 in the distal section 204 to expose a conducive portion of the lead conductor 228. For example, the cover member 230 is exposed, such as ablated or skived, along with the part of the electrically insulative sheath of the lead conductor 228 to expose bare wire of the conductive lead along the side of the shaft 202.

The electrode 226 in the illustrated embodiment is formed as a conductive ring element to encircle the cover member 230. The ring can be contiguous or noncontiguous. In one example, the ring electrode includes an inner diameter slightly larger than an outer diameter of the outer surface 232 of the cover member 230 to fit over the cover member 230 during manufacture. The electrode 226 is crimped or swaged over the cover member 230 to make electrical contact with the exposed conductive portion of the associated lead conductor 228 via mechanical compression during manufacture. In one embodiment, the mechanical compression provides the only mechanical connection between lead conductor 228 and the electrode. The mechanical compression provides a robust electrical connection between the electrode 226 and the lead conductor 228, and the electrical connection is made without potential leak paths in the support member 220 or a laser weld attaching the electrode 226 to the lead conductor 228. In other embodiments, the mechanical compression and a laser weld is provided to enhance the electrical connection.

The mechanical compression of the swaged electrode 226 can include the possibility of lead conductor 228 or electrode 226 contacting the conductive braided member of the support member 220, which could cause an electrical short between the electrode 226 and the support member 220. To mitigate this possibility, the shaft 202 can include an electrically insulative sleeve 236 disposed between the support member 220 and the conductive lead 228 in the region radially under the electrode 226. In the illustrated example, the electrically insulative sleeve 236 includes an axial length that is slightly longer than an axial length of the electrode 226 such that portions of the sleeve 236 extend from underneath the electrode 226. In one embodiment, the sleeve is a heat shrink polyethylene terephthalate (PET). The heat shrink PET sleeve 236 is disposed on the outer surface 222 of the support member 220 prior to the lead conductor 228 wound around the outer surface 222 of the support member 220 and the sleeve 236. In other embodiments, the sleeve can be constructed from an ultraviolet curable adhesive barrier or a polyimide barrier. In still other embodiments, the sleeve is a longer length or full length tube, ribbon, or braid of polyether block amide such as sold under the trade designation PEBAX. Other mitigations include forming a non-conductive support member 220, employing thicker insulative sheath on the lead conductor 228, and applying thicker cover member 230. In some embodiments, the electrode is disposed distal to the support member, such as in the case of a electrode distal to a pull ring in which the distal end of the support member terminates at the pull ring. In such an example, the conductors can be extended, such as coiled, distal to the support member, such as the braided member, and extended to the electrode.

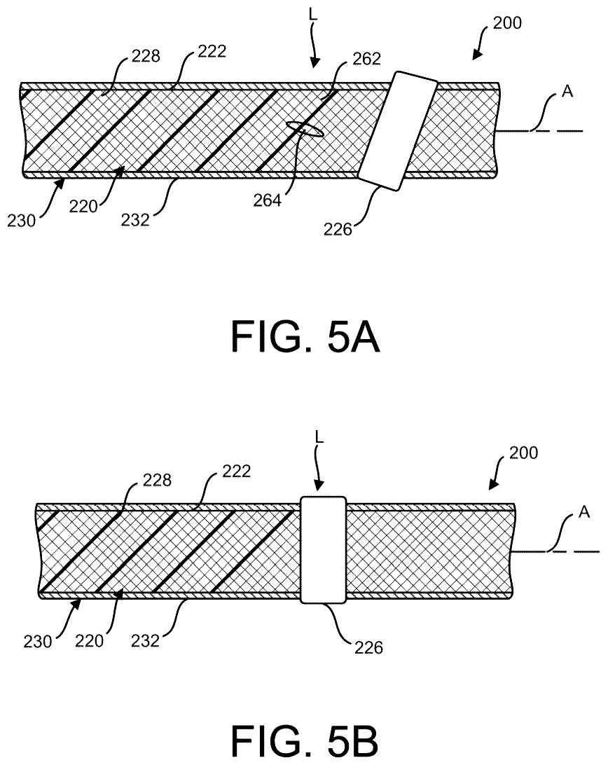

FIG. 4 illustrates an example method 400 of manufacturing the sheath 200. A shaft having a lead conductor wound around an outer surface of a support member is provided at 402. A cover member is formed as a coating of an electrically insulative material over the support member and conductive leads at 404. The lead conductor is selectively exposed, such as skived, through the cover member at the desired axial location on the shaft for the electrode associated with the lead conductor at 406. The electrode is positioned over the skived lead conductor and is swaged on the shaft to make an electrical connection with the lead conductor via mechanical compression against the shaft at 408.

In one embodiment, the method 400 includes manufacturing a sheath shaft having a lead conductor wound around an outer surface of a support member at 402. In one example, the lead conductor is wound on a fully formed support member. In another example, the lead conductor is formed into the support member as braided fibers in the woven fabric. In one embodiment, the number of lead conductors corresponds with a number of electrodes to be attached to the shaft. In one embodiment, the shaft includes four longitudinally spaced-apart electrodes and four associated lead conductors. In embodiments, the lead conductors are coiled or braided together along the outer surface of the support member. The amount of pitch of the wound lead conductor approximates that of the braided support member to reduce buckling during deflection of the sheath. In one example, the lead conductor is wound at a pitch less than that of the braided fabric of the support member. The lead conductors can be coiled manually with a slit rubber silicon sheet to hold lead conductors uniformly apart. Also, the lead conductors can be coiled with a multi-filar coiler, and braided lead conductors can be formed with a multi-bobbin (four bobbins) conductor. In one embodiment, a sacrificial or dummy mandrel formed from stainless-steel or a thermoplastic, for example, can be braided tri-axially or under the outermost braid of the support member to provide heat shielding and protect the main lumen of the shaft from additional manufacturing processes.

In one embodiment, an electrically insulative sleeve is disposed on support member prior to the lead conductor being wound on the outer surface at 402. The sleeve can be applied to support members having conductive fibers, such as a stainless-steel braided fabric. The sleeve, such as a PET heat shrink sleeve, is positioned at select axial locations of the support member that will be radially underneath later-attached electrodes. As an alternative to the sleeve, the lead conductor can include a thicker insulative sheath over the wire, a non-conductive support member or braided member can be employed, the braid pattern can be modified, the outer diameter of the support member can be reduced, wall thickness of the electrode can be reduced, or less force can be applied at swaging the electrode ring.

A cover member is formed as a coating of an electrically insulative reflowable plastic or thermoplastic material that extends over the support member and conductive leads and seals underlying components of the shaft at 404. In one example, the distal and proximal ends of the support member can be sealed to prevent the material from entering the lumen, and the lead conductor can be attached to the support member in preparation for the cover to hold the lead conductor in place.

The lead conductor is selectively skived at the desired axial location on the shaft for the electrode associated with the lead conductor at 406. In one example, the lead conductor is ablated, such as with a laser, at the desired axial location. Each lead conductor is selectively skived at the desired axial location on the shaft for the associated electrode. In one example, the cover member and sheath of the lead conductor are removed with a razor blade to expose the bare wire of the lead conductor on the shaft. In another example, a laser is applied to ablate the cover member and the sheath of the lead conductor such as via machine vision.

The electrode is positioned over the exposed bare wire and is swaged to make an electrical connection with the lead conductor via mechanical compression against the shaft at 408. A ring electrode having an inner diameter slightly larger than an outer diameter of the cover member at the skived location is compressed onto the cover member to make a robust electrical connection with the lead conductor.

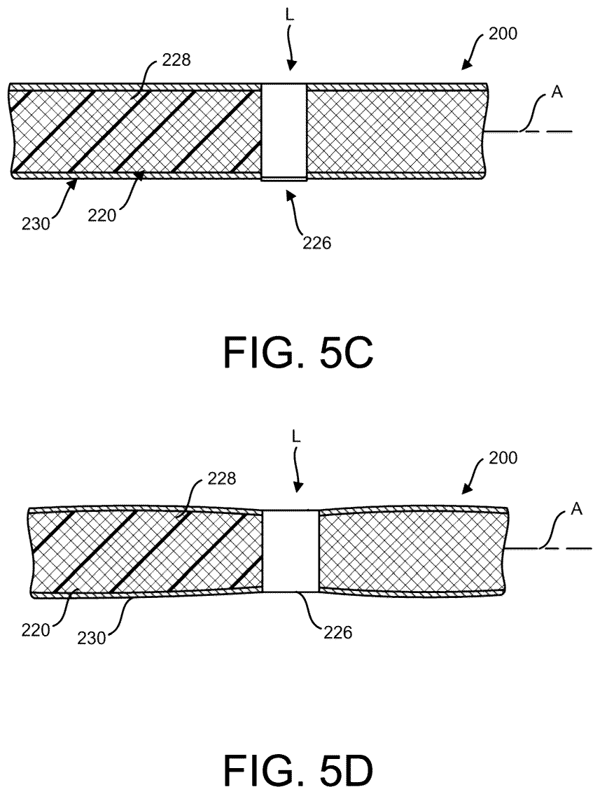

FIGS. 5A-5D illustrate manufacture of the sheath 200 according to method 400 after the lead conductor 228 has been wound around the support member 220 at 402 and the cover member 230 has been applied at 404. FIG. 5A illustrates a ring electrode 226 having an inner diameter slightly larger than the outer diameter of the cover member 230 but out of axial position as the lead conductor 228 is selectively skived at the desired axial location L on the shaft 202 for the electrode 226 such as at 406. In the illustrated example, the cover member 230 and sheath 262 of the lead conductor 228 are removed to expose the bare wire 264 of the lead conductor 228. FIG. 5B illustrates the ring electrode 226 moved into position and placed over the exposed bare wire 264 at desired axial location L. FIG. 5C illustrates the electrode 226 after the electrode 226 has been crimped against the cover member 230 to hold the electrode in position over the exposed bare wire 264, and FIG. 5D illustrates the electrode 226 after the electrode has been skived at the desired location L to be compressed on the cover member 230 to make a robust electrical connection with the lead conductor 228 at the bare wire 264 such as at 408. In the illustrated embodiment, the skived electrode includes an inner diameter that is less than the outer diameter of the cover member prior to positioning the electrode 226. The application of force to the electrode in FIGS. 5C and 5D can be with separate tools. In one example, the electrode is crimped or swaged with one manufacturing movement rather than multiple movements at 408.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A sheath, comprising:
an elongated shaft defining a lumen along a longitudinal axis and having a distal section opposite a proximal section, the elongated shaft having an outermost elongated support member coaxial with the lumen, the support member having an outer side;
a lead conductor wound around the outer side of the support member at the distal section;
a cover member disposed on the lead conductor and the support member, the cover member and lead conductor, a conductive portion of the lead conductor exposed at a selected axial location in the distal section; and
an electrode swaged against the cover member at the selected axial location and in electrical connection with the exposed conductive portion via mechanical compression.

2. The sheath of claim 1, wherein the support member includes a braided fabric comprising a plurality of fibers braided together.

3. The sheath of claim 1, wherein the lead conductor is wound on the outer side of the support member.

4. The sheath of claim 3, wherein the lead conductor is coiled on the outer side of the support member.

5. The sheath of claim 3, wherein the lead conductor includes a plurality of lead conductors, and the plurality of lead conductors are braided on the outer side of the support member.

6. The sheath of claim 2, wherein the lead conductor is a fiber of the plurality of fibers braided together to form the braided fabric.

7. The sheath of claim 1, wherein the support member is conductive and further comprising an electrically insulative sleeve disposed on the support member and radially underneath the exposed conductive portion.

8. The sheath of claim 7, wherein the electrically insulative sleeve is a polyethylene terephthalate heat shrink.

9. The sheath of claim 1, wherein the lead conductor includes a conductive wire covered in an electrically insulative sheath member, and the exposed conductive portion includes a bare wire portion.

10. The sheath of claim 1, wherein the lead conductor includes a conductive element having a triangular cross section with a vertex facing radially outwardly and a side facing radially inwardly.

11. The sheath of claim 1, wherein the electrode is ring electrode.

12. The sheath of claim 11, wherein the ring electrode includes an inner diameter, and the cover member includes an outer diameter, wherein the inner diameter is greater than the outer diameter prior to the electrode being swaged against the cover member.

13. The sheath of claim 11, wherein the ring electrode is a contiguous ring electrode.

14. The sheath of claim 1, wherein the shaft includes one support member.

15. The sheath of claim 1, wherein the electrode is not welded to the lead conductor.

16. The sheath of claim 1, wherein the sheath is a guide sheath having a proximal end coupled to a handle.

17. A sheath, comprising:
an elongated shaft defining a lumen along a longitudinal axis and having a distal section opposite a proximal section, the elongated shaft having an outermost elongated support member coaxial with the lumen, the support member having an outer side;
a lead conductor wound around the outer side of the support member at the distal section;
a cover member disposed on the lead conductor and the support member, the cover member and lead conductor skived at a selected axial location in the distal section to expose a conducive portion of the lead conductor; and
an electrode swaged against the cover member at the selected axial location and in electrical connection with the exposed conductive portion via mechanical compression.

18. The sheath of claim 17, wherein the support member includes a braided fabric comprising a plurality of fibers braided together, and wherein the lead conductor is one of wound on the outer side of the support member and the lead conductor is a fiber of the plurality of fibers braided together to form the braided fabric.

19. A method of forming a sheath, the method comprising:
providing an elongated shaft defining a lumen along a longitudinal axis
and having a distal section opposite a proximal section, the elongated shaft having an outermost elongated support member coaxial with the lumen, the support member having an outer side;
winding a lead conductor around the outer side of the support member at the distal section;
disposing a cover member disposed on the lead conductor and the support member;
exposing a conductive portion of the lead conductor at a selected axial location in the distal section; and
swaging an electrode against the cover member at the selected axial location and in electrical connection with the exposed conductive portion via mechanical compression.

20. The method of claim 19, wherein the cover member and lead conductor are skived to expose the lead conductor at the selected axial location in the distal section.
